(19) United States
(12) Patent Application Publication
(10) Pub. No.: US 2004/0107515 A1
(43) Pub. Date: Jun. 10, 2004

(54) Hair colouring compositions and their use

(75) Inventors: Dominic Pratt, Darmstadt (DE); James Charles Dunbar, Morrow, OH (US); Delyth Angharad James, Egham (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/679,776

(22) Filed: Oct. 6, 2003

Related U.S. Application Data
(63) Continuation of application No. PCT/US02/10625, filed on Apr. 4, 2002.

(30) Foreign Application Priority Data
Apr. 5, 2001 (GB) 0108615.6

(51) Int. Cl. A61K 7/13

(52) U.S. Cl. 8/405

(57) ABSTRACT

A hair colouring composition comprising one or more developers is selected from amino aromatic systems capable of being oxidised and thereafter undergoing at least two electrophilic attach reactions, and one or more couplers selected from (a) 1,3-diketones of the formula (I): in which X and Y are non-leaving substituents and Z is an active leaving group, such that in the presence of an oxidising agent the or each developer reacts with the or each coupler substantially only at the position having the active leaving group Z and (b) compoounds of the formula (II): in which R1, R2 and R3 are, independently, cycloalkyl, alkenyl, cycloalkenyl, aryl, alkaryl, aralyl, —R′NHCOR, —CONHR, —R′CONHR, —R′OH, —R′SO2R, —R′SO2NHR, —R′OR or —COR, in any of which R is H, alkyl, cycloalkyl, alkenyl, cycloalkenyl, aryl, alkaryl, or aralkyl and R′ is alkylene, alkenylene, cycloalkylene, cycloalkenylene, arylene, alkarylene or aralkyrene, or substituted versions of any of these.

### CROSS REFERENCE TO RELATED APPLICATION

The application is a continuation of International application PCT/US02/10625 (Case CM2530F) filed on Apr. 4, 2002.

This invention relates to new compositions for colouring hair and to methods of using the compositions in hair colouring processes. In particular it relates to new combinations of developer and coupler components.

Such compositions comprise various aromatic compounds, commonly known as developers (also known as precursors or primary intermediates), together with various other compounds, commonly known as couplers. These are referred to as oxidative hair colouring agents because they require an oxidising agent for formation of colour.

In our earlier publications WO98/52519, WO98/52520, WO98/52521, WO98/52522 and WO98/52523 we describe specific hair colouring compositions which use defined types of oxidative hair colouring agents. In particular, in these publications it is essential that the couplers are selected from three groups (A), (B) and (C), which give cyan, yellow and magenta colours respectively when coupled with a developer. The “yellow” couplers (B) are 1,3-diketones containing the group —C(O)CHZC(O)— in which Z is an active leaving group. It is also essential that the composition comprises at least one developer selected from amino aromatic systems capable of being oxidised and thereafter undergoing a single electrophilic attack (single electrophilic attack developers). According to these publications the combination of the chosen couplers and this specific type of developer has the advantage that the reaction chemistry is closely defined, because each developer molecule reacts only once with a single coupler molecule. Thus it is possible to predict with precision the compounds which will be present in the hair at the end of the colour forming reaction.

GB 1,025,916 also describes combinations of developers and couplers of this general type, but itself has disadvantages overcome by the international publications discussed above.

To our knowledge, the 1,3-diketone couplers of this type have been described only in combination with the developers of the single electrophilic attack type.

We have found that although these compositions have significant advantages, there is room for improvement. In particular, we find that the colours produced, although vibrant and easily predictable, tend to give higher fade under the influence of ultraviolet light (UV fade) than would be desirable. Thus when the consumer's hair has been coloured, over a period of weeks there is a noticeable fading in the colour, if exposed to a substantial amount of UV.

It would be desirable to improve the UV fade resistance properties of compositions which contain the 1,3-diketone couplers.

According to the invention we provide a hair colouring composition comprising one or more developers selected from amino aromatic systems capable of being oxidised and thereafter undergoing at least two electrophilic attack reactions, and one or more couplers selected from 1,3-diketones of the formula I:

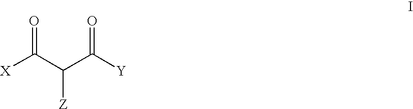

in which X and Y are non-leaving substituents and Z is an active leaving group, such that in the presence of an oxidising agent the or each developer reacts with the or each coupler substantially only at the position having the active leaving group Z and

compounds of the formula II:

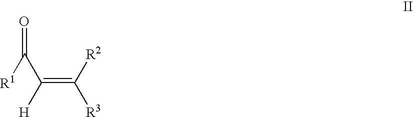

in which R₁, R₂ and R₃ are, independently, cycloalkyl, alkenyl, cycloalkenyl, aryl, alkaryl, aralkyl, —R′NHCOR, —CONHR, —R′CONHR, —R′OH, —R′SO₂R, —R′SO₂NHR, —R′OR or —COR, in any of which R is H, alkyl, cycloalkyl, alkenyl, cycloalkenyl, aryl, alkaryl or aralkyl and R′ is alkylene, alkenylene, cycloalkylene, cycloalkenylene, arylene, alkarylene or aralkyrene, or substituted versions of any of these.

The invention also provides a hair colouring kit comprising the defined developer and the defined coupler separately packaged.

The invention also provides a method for colouring hair comprising applying to the hair the defined developer and the defined coupler.

We find surprisingly that the choice of developers which are not the single electrophilic attack developers essential in the earlier international publications and GB 1,025,916 significantly improves the UV fade resistance of the resulting colour.

Consequently, the invention also provides the use of the defined developers to improve the UV fade resistance of compositions comprising the defined developers.

It is unclear precisely why this benefit arises, but nevertheless it is a demonstrable benefit, as will be seen below.

This invention is also applicable to a further known class of couplers which give a yellow colour, having the formula II above.

In the invention the developer is an amino aromatic compound which has a structure such that it is capable of being oxidised by an oxidising agent. The structure is also such that the oxidised developer is capable of undergoing electrophilic attack on at least two other molecules. Suitable developers of this type include aminoaromatic systems in which there are two primary amine groups.

The coupler is of a specific formula which has an active leaving group Z at a defined site. By an “active leaving group” we mean any group which can be removed (under the conditions prevailing during the hair-dyeing process) so that the developer reacts at that position in the coupler molecule. A bond between the coupler and developer molecule is thus formed at the site of the active leaving group. Examples of active leaving groups are H, PhO, Cl, Br, alkoxy (RO) such as phenoxy PhO, and RS— in which R is alkyl or aryl, but any leaving group which leaves during the reaction so as to allow coupling between developer and coupler is suitable.

The couplers are 1,3-diketones of the formula I. They are of the same general type as disclosed in WO98/52519, WO98/52520, WO98/52521, WO98/52522 and WO98/52523 as yellow couplers. The couplers produce a yellow colour on reacting with a developer molecule. The defined coupler may be the only coupler in the composition but preferably other types of coupler are also used to give a range of different colours. Each of X and Y may be independently, H, alkyl, cycloalkyl, alkenyl, cycloalkenyl, aryl, alkaryl, aralkyl, —R′NHCOR, —R′CONHR, —ROH, —R′SO₂R, —R′CO₂NHR, —NHCOR, —NR₂, —NHR, —NH₂, —R′OR and —OR. In these groups R can be H, alkyl, cycloalkyl, alkenyl, cycloalkenyl, aryl, alkaryl or aralkyl and R′ is alkylene, cycloalkylene, alkenylene, cycloalkenylene, arylene, alkarylene or aralkylene. Substituted versions of any of these can be used. Suitable substituents include OH, —OR, Cl, Br, F, —CO₂H, —CO₂R, —NH₂ and —COR.

The coupler may also be selected from compounds of the formula H but is preferably selected from compounds of the formula I.

In the formula I, X and Y are each preferably methyl or ethyl and preferred couplers of the formula I are dimethyl acetoacetamide and diethyl acetoacetamide.

The amount of coupler is often from 0.05 to 4 wt. %, eg 0.05 to 3 wt. %, but in some cases is up to 5 or 6%. For instance it may be from 0.2 to 3 wt. %, eg 0.2 to 2 wt. %, based on the composition.

The developer is one which is capable of being oxidised and undergoing at least two electrophilic attack reactions. Preferably it is selected from para and ortho substituted benzene compounds, disubstituted pyridine compounds, disubstituted pyrimidines and diamino substituted pyrazoles. Preferably it is paraphenylene diamine.

Developer is often included in the composition in amounts of from 0.01 to 7%, eg 0.01 to 5%, by weight based on total composition applied to the hair. Preferred amounts of developer are from 0.3 to 4%, eg 0.3 to 2%, preferably 0.4 to 3%, eg 0.4 to 1.5%.

Preferably the composition is substantially free of developers which are capable of being oxidised and thereafter undergoing only a single electrophilic attack.

For both developers and couplers the solubility properties can be important. The developer and coupler compounds themselves should have solubility such that they can be formulated in appropriate concentrations. They preferably have solubility of at least 5 g/100 ml, deionised water at pH about 10 and 25° C.

The composition may contain any other suitable optional ingredients. It may contain other couplers of the types disclosed in WO98/92522 and the PCT publications discussed above, as well as standard oxidative couplers and colouring components.

An optional component of the composition is an antioxidant. It may be selected from for instance sulphites such as sodium sulphite, hydroquinone, sodium bisulphite, sodium metabisulphite, thioglycolic acid, sodium dithionite, erythrobic acid and other mercaptans, ascorbic acid and n-propyl gallate.

If used, antioxidant is included in an amount of at least 0.01 and usually not more than 3 or 4 wt. %, based on total weight of composition applied to the hair.

For the developers and couplers to be effective in forming colour they require the presence of an oxidising agent. This oxidising agent is normally included in the composition just before it is applied to the hair. Normally the composition of the invention will be supplied in at least two individual packages such as bottles, the oxidising agent being included in one package and the developers and couplers being included in another.

A preferred oxidising agent is hydrogen peroxide. This is often used in amounts up to 10 wt. %, based on composition applied to the hair. Other oxidising agents which may be used include other inorganic peroxygen oxidising agents, preformed organic peroxyacid oxidising agents and other organic peroxides such as urea peroxide, melamine peroxide, and mixtures of any of these.

Suitable oxidising agents are preferably water-soluble, that is they have a solubility of at least about 5 g in 1,000 ml of deionised water at 25° C. (“Chemistry” C. E. Mortimer, 5th Edition, page 277).

Usually the colouring compositions of the invention have pH above 7, in particular above pH 8 or 9. A pH of from 9 to 12 is often suitable. The systems of the invention can also be incorporated into low pH (eg pH 1 to 6) hair colouring systems.

In practice the composition of the first aspect of the invention may be supplied to the consumer as a single package containing developer and coupler in a single unit such as a bottle.

It is also possible to supply the composition so that the developers are individually packaged and the couplers are individually packaged. Couplers may be supplied as a preformed mixture selected to give a particular colour. Alternatively they can be supplied separately for mixing by the consumer to give a variety of different hair colours.

In all cases, the essential components are mixed to form the composition of the invention before application to the hair.

Generally oxidising agent is individually packaged separately from any of the colouring components. It is often mixed with these to form a component of the hair colouring composition before application to the hair. Alternatively it can be applied to the hair separately either before or after the hair colouring composition.

The developers, couplers and oxidising agent, and any other materials to be applied to the hair as components of the composition of the invention, may be provided in any suitable physical form. A preferred physical form is liquid. The liquid may be of low viscosity, for instance it may be water thin, or it may be of higher viscosity. The material may be suspended in a gel network. The gel may be solid or of low viscosity.

The materials for colouring the hair are often formulated so that when they are mixed to form the composition of the invention for application to the hair they form a product of cream-like consistency, which is convenient for application to the hair. The final composition which is applied to the hair is often in the form of an emulsion.

Each individual material may be supplied in a form such that the composition containing it has a pH of above or below 7. For instance it may be from pH 1 to 11. In order to assist solubility of the various components, particularly developers and couplers, in a water-based carrier, the carrier may have a pH of above 6.1 or 6.5 or even above 7, for instance from pH 8 or 9 to pH 10 or 11. A pH as supplied of from 1 to 6 can assist in improving stability of the components.

The materials may be provided such that the pH of the final composition when mixed for application to the hair has a pH below 7 even though one of the components used to form it has a pH of above 7. Alcohols such as ethanol in amounts of from for instance 5 to 10 or 25% may be included to aid solubility of the developers and, particularly, the couplers in a water-based carrier.

Suitable optional ingredients include other oxidative and non-oxidative colouring agents, buffering agents, hair swelling agents, catalysts for the oxidising agent, thickeners, diluents, enzymes, surfactants (especially anionic, non-ionic amphoteric and zwitterionic surfactants), proteins and polypeptides and derivatives thereof; water-soluble or solubilizable preservatives; dye removers; H₂O₂ stabilisers; moisturising agents; solvents; anti-bacterial agents; low temperature phase modifiers; viscosity control agents; hair conditioning agents; enzyme stabilisers; TiO₂ and TiO₂-coated mica; perfumes and perfume solubilizers; chelating agents. Other optional materials include anti-dandruff actives such as ZPT. Details of suitable optional ingredients can be found in WO98/52522.

### EXAMPLES

The invention will now be illustrated by the following examples. In these examples, various standard tests are used, as follows.

I Assessment of Initial Colour and Colour Change (Measurement of AE)

The equipment used to measure both the initial colour and colour change of substrates (hair/skin) dyed with colouring compositions of the present invention is a Hunter Colourquest spectrophotometer. The value used to express the degree of colour change on any particular substrate is Delta E (ΔE). Delta E, as defined herein, is represented by a factual sum of L, a, and b values such that:

ΔE=(ΔL²+Δa²⁺Δb²)^{1/2}

and L is measure of lightness and darkness (colour intensity), wherein L=100 is equivalent to white, and L=0 is equivalent to black. Further, “a” is a measure of the red and green quotients (colour hues) such that positive equates to red and negative to green and “b” is a measure of the yellow and blue quotients (colour hues) such that positive equates to yellow and negative equates to blue.

Hunter Colourquest measurements can be carried out on the Hunter Labscan Colourimeter which is a full scanning spectrocolorimeter with a wavelength of from 400-700 nanometers which records the colour of test hair switches (tresses) in terms of “L”, “a” and “b” values. The machine is set to: mode—0/45; port size—1 inch; view size—1 inch; light—D65; field of view—10°; UV lamp/filter—none. The hair is placed in a sample holder designed to hold the hair in uniform orientation during measurement. Equivalent colorimeters can be used, but it must be ensured that the hair does not move during measurement. The hair must be spread to cover the 1 inch port during colour measurement. Dots are placed on the switch holder to guide the positioning of the holder at the port. The dots are lined up with a mark on the port and readings are taken at each spot.

Eight measurements are run per switch, 4 on each side, and three switches are run per treatment.

II Standard Hair Switch

The compositions according to the present invention can be used to colour hair of all colours, types and condition. For the purposes of illustration various test hair switches can be tested herein. Two of these standard hair switches can be measured in terms of their approximate L, a, b values.

| | L | a | b |
|---|---|---|---|
| Light brown | about 60 | about 9 | about 32 |
| (permed and bleached) | | | |
| 40% grey dark brown | 35-37 | 4.5-5.5 | 11.5-12.7 |

Yak hair (virgin or permed and/or bleached) can also be used. It has values of: L=about 82 to 83, a=about −0.5 to 0.7, b=about 11 to 12.

III Hair Switch Colouring Method

To colour hair, a 4 gramme switch of about 8 inch long hair (or a 2 gramme switch of 4 inch long hair) is hung over a suitable container. The test colouring product is then prepared (ie, where applicable the separate bottle components are mixed together) and about 2 grammes of product per gramme hair is applied directly to the test hair switch. The colourant is massaged through the hair switch for up to about 1 minute and then left on the hair switch for up to about 30 minutes. After rinsing with running water for about 1 or 2 minutes the coloured hair switch is then cleansed (according to the shampoo protocol IV below) and dried. Drying can be effected either naturally (without heat assistance) or using a drier. The colour development (initial colour) of the coloured, cleansed, dried test hair switch can then be assessed using the Hunter Colourquest spectrophotometer.

IV Hair Switch Cleansing Method

A 4 gramme, 8 inch test switch (or a 2 gramme, 4 inch test switch) of coloured hair is clamped over a suitable container and rinsed thoroughly for about 10 seconds using warm water (at about 100° F. at about 1.5 gallons/minute pressure). Shampoo (about 0.1 ml non-conditioning shampoo per gramme hair) can then be applied directly to the wet test switch using a syringe. After lathering the hair for about 30 seconds the hair is rinsed in running water for about 30 seconds. The shampoo and lathering process is then repeated with a final 60 second rinse. Excess (water can be removed (squeezed) from the test switch using the fingers. The test switch is then dried either naturally, or using a pre-heated dryer box at about 140° F. (for about 30 minutes).

V Perming Protocol

The following method is used to perm hair which is usually subsequently to be bleached.

A 4 gram switch of about 8 inch long hair is hung over a suitable container. Perming solution supplied under the trade name “Zotos” is applied to the hair so as to saturate it totally.

The switches are then resaturated. The switches are then laid on a plastic tray for 20 minutes and subsequently rinsed for 1½ to 2 minutes with tap water at 37° C. The switches are squeezed dry and towelled dry. The switches are then hung over the container again and commercially available “Zotos” neutraliser is applied so as to saturate them. They are then laid in the plastic tray for 5 minutes and subsequently rinsed for 1½ to 2 minutes in tap water at 37° C. The switches are then shampooed twice and left to dry.

### Example 1

Hair switches were coloured with the coupler and developer system specified below, washed and dried. Samples were then subjected to 21 hours in a Xenotester. E=44 W/m² (equivalent to 7 days European sunlight) @ 300-400 nm, 65% Relative humidity, 40-50° C., turning mode. Lab measurements were recorded before and after exposure for a delta E fade to be calculated.

The formulation used comprised the specified developer in combination with yellow couper N,N-dimethylacetoacetamide, ascorbic acid, EDTA, sodium sulphate, ammonium acetate, ammonium hydroxide, hydrogen peroxide, water and emulsion base. The formulation was applied to the hair switches for 30 minutes at 30° C. unwrapped.

Results are shown in Table 2 below.

TABLE 2

| | | | | delta E |
|---|---|---|---|---|
| Combination | L | a | b | change |
| Before virgin: PPD + N,N- | 25.69 | 6.56 | 12.57 | |
| dimethylaceto acetamide | | | | |
| After virgin: PPD + N,N- | 30.77 | 5.32 | 16.66 | 6.64 |
| dimethylaceto acetamide | | | | |
| Before permed: PPD + N,N- | 23.74 | 8.15 | 10.12 | |
| dimethylaceto acetamide | | | | |
| After permed: PPD + N,N- | 25.82 | 7.01 | 11.15 | 2.59 |
| dimethylaceto acetamide | | | | |
| Before virgin: | 53.85 | 3.02 | 44.72 | |
| dichloropara aminophenol + N,N- | | | | |
| dimethylacetoacetamide | | | | |
| After virgin: dichloropara | 57.83 | 7.19 | 49.81 | 7.69 |
| aminophenol + N,N- | | | | |
| dimethylacetoacetamide | | | | |
| Before permed: dichloropara | 54.10 | 1.28 | 39.57 | |
| aminophenol + N,N- | | | | |
| dimethylacetoacetamide | | | | |
| After permed: | 57.06 | 4.66 | 47.01 | 8.69 |
| dichloropara aminophenol + N,N- | | | | |
| dimethylacetoacetamide | | | | |

The data shows that PPD gives a consumer noticeable UV fade benefit versus dichloroparaminophenol as a developer in combination with the yellow coupler.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A hair colouring composition comprising:
a) one or more developers selected from amino aromatic systems capable of being oxidised and thereafter undergoing at least two electrophilic attack reactions; and
b) one or more couplers selected from the group consisting of:
i) 1,3-diketones of the formula (I): 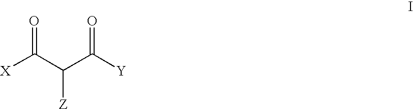
wherein X and Y are non-leaving substituents and Z is an active leaving group, such that in the presence of an oxidising agent the or each developer reacts with the or each coupler substantially only at the position having the active leaving group Z; and
ii) compounds of the formula (II): 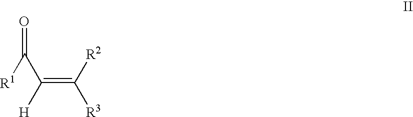
wherein R₁, R₂, and R₃ are independently selected from the group consisting of cycloalkyl, alkenyl, cycloalkenyl, aryl, alkaryl, aralkyl, —R′NHCOR, —CONHR, —R′CONHR, —R′OH, —R′SO₂R, —R′SO₂NHR, —R′OR, and —COR;
wherein R is selected from the group consisting of H, alkyl, cycloalkyl, alkenyl, cycloalkenyl, aryl, alkaryl, and aralkyl; and
wherein R′ is selected from the group consisting of alkylene, alkenylene, cycloalkylene, cycloalkenylene, arylene, alkarylene, and aralkyrene, and substituted versions of any of these.

2. A hair colouring composition according to claim 1, wherein the couplers are selected from compounds of the formula (I).

3. A composition according to claim 1, wherein the composition comprises substantially no developer which is an amino aromatic system capable of being oxidised and thereafter undergoing a single electrophilic attack.

4. A composition according to claim 1, wherein the coupler is dimethyl acetoacetamide.

5. A composition according to claim 1, wherein the coupler is diethyl acetoacetamide.

6. A composition according to claim 1, wherein the one or more developers are selected from the group consisting of para and ortho disubstituted benzene compounds, disubstituted pyridine compounds, disubstituted pyrimidines, and diamino substituted pyrazoles.

7. A composition according to claim 6, wherein the developer is para-phenylene diamine.

8. A method of improving UV fade resistance of a colour delivered by a composition for colouring hair, said method comprising the steps of:
a) selecting one or more developers from amino aromatic systems capable of being oxidised and thereafter undergoing at least two electrophilic attack reactions; and
b) selecting one or more couplers from the group consisting of:
i) 1,3-diketones of the formula (I): 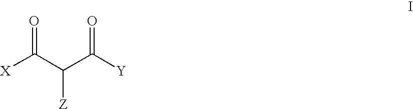
wherein X and Y are non-leaving substituents and Z is an active leaving group, such that in the presence of an oxidising agent the or each developer reacts with the or each coupler substantially only at the position having the active leaving group Z; and
ii) compounds of the formula (II): 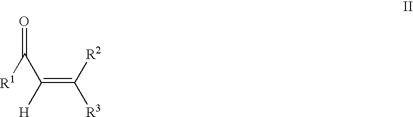
wherein R₁, R₂, and R₃ are independently selected from the group consisting of cycloalkyl, alkenyl, cycloalkenyl, aryl, alkaryl, aralkyl, —R′NHCOR, —CONHR, —R′CONHR, —R′OH, —R′SO₂R, —R′SO₂NHR, —R′OR, and —COR;
wherein R is selected from the group consisting of H, alkyl, cycloalkyl, alkenyl, cycloalkenyl, aryl, alkaryl, and aralkyl; and
wherein R′ is selected from the group consisting of alkylene, alkenylene, cycloalkylene, cycloalkenylene, arylene, alkarylene, and aralkyrene, and substituted versions of any of these.

9. A method according to claim 8, wherein the composition for colouring hair contains at least one coupler selected from compounds of the formula (I).

10. A method according to claim 8, wherein the developer is selected from the group consisting of para and ortho disubstituted benzene compounds, disubstituted pyridine compounds, disubstituted pyrimidines, and diamino substituted pyrazoles.

11. A method according to claim 10, wherein the developer is para-phenylene diamine.

12. A method according to claim 8, wherein the composition comprises substantially no developer which is an amino aromatic system capable of being oxidised and thereafter undergoing a single electrophilic attack

13. A method according to claim 8, wherein the coupler is dimethyl acetoacetamide.

14. A method according to claim 8, wherein the coupler is diethyl acetoacetamide.

15. A method for colouring hair comprising:
a) applying to the hair one or more developers selected from amino aromatic systems capable of being oxidised and thereafter undergoing at least two electrophilic attack reactions; and
b) applying one or more couplers selected from the group consisting of:
i) 1,3-diketones of the formula (I): 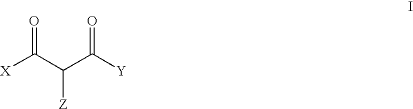
wherein X and Y are non-leaving substituents and Z is an active leaving group, such that in the presence of an oxidising agent the or each developer reacts with the or each coupler substantially only at the position having the active leaving group Z; and
ii) compounds of the formula (H): 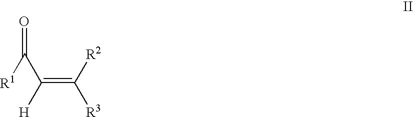
wherein R₁, R₂, and R₃ are independently selected from the group consisting of cycloalkyl, alkenyl, cycloalkenyl, aryl, alkaryl, aralkyl, —R′NHCOR, —CONHR, —R′CONHR, —R′OH, —R′SO₂R, —R′SO₂NHR, —R′OR, and —COR;
wherein R is selected from the group consisting of H, alkyl, cycloalkyl, alkenyl, cycloalkenyl, aryl, alkaryl, and aralkyl; and
wherein R′ is selected from the group consisting of alkylene, alkenylene, cycloalkylene, cycloalkenylene, arylene, alkarylene, and aralkyrene, and substituted versions of any of these.

16. A hair colouring kit comprising:
a) an individually packaged colouring component comprising:
i) one or more developers selected from amino aromatic systems capable of being oxidised and thereafter undergoing at least two electrophilic attack reactions; and
ii) one or more couplers selected from the group consisting of compounds of the formula (I) and compounds of the formula (II); and
b) an individually packaged oxidising component.
